(19) United States
(12) Patent Application Publication
(10) Pub. No.: US 2021/0259377 A1
(43) Pub. Date: Aug. 26, 2021

(54) HYGIENICAL CASE FOR REMOTE CONTROLS

(71) Applicants: Luca RAMONDA, Moruzzo (UD) (IT); Georgios MAVRIDIS, Trieste (IT); William BELLINA, Porcia (PN) (IT); Massimo CRAPIS, Udine (IT)

(72) Inventors: Luca RAMONDA, Moruzzo (UD) (IT); Georgios MAVRIDIS, Trieste (IT); William BELLINA, Porcia (PN) (IT); Massimo CRAPIS, Udine (IT)

(21) Appl. No.: 17/053,978

(22) PCT Filed: May 17, 2019
(86) PCT No.: PCT/IB2019/054087

§ 371 Date: Nov. 9, 2020

(30) Foreign Application Priority Data
May 18, 2018 (IT) 102018000005505

(51) Int. Cl. A45C 11/00; A61L 2/16

(52) U.S. Cl. CPC A45C 11/00; A61L 2/16

(57) ABSTRACT

A hygienical case for a remote control is described. The hygienical case has a first wrapper adapted to cover totally, or substantially totally, a remote control, and a second wrapper fixed externally to the first wrapper so as to cover totally, or substantially totally, the first wrapper. The second wrapper is permanently removable from the first wrapper.

The present invention refers to a hygienical case for remote controls.

Currently, in commonly used structures, i.e. adapted for being used by a plurality of users, like for example hotel rooms, hospitals, etc., it is known to use remote controls, wired or wireless, for remotely controlling electric or electronic devices, such as television sets, radios, bed tilting devices, curtain openers/closers, air conditioning systems, etc.

Such remote controls are typically handled by users that take turns in such communal structures, with the risk, if such remote controls are not properly cleaned or hygienised before being used by a subsequent user, of possible germs, bacteria, viruses or other pathogens that may be deposited on the remote control during the previous use, being transmitted to the next user. Known studies confirm that the area that has the greatest bacterial contamination in hotel rooms is indeed the television remote control.

In order to try to avoid such problems, there is for example the known Italian patent application number MO2012A0000282, which describes a case for remote controls comprising a coating wrapper for coating substantially the entire remote control provided with at least one keypad, the coating wrapper comprising at least one sector that is at least partially transparent and can be arranged at the keypad of the remote control to allow the keypad to be seen; the case comprises at least one covering element, which is removably associated with the coating wrapper substantially at the sector, and is adapted to prevent the keypad being seen until the covering element has been removed from the coating wrapper.

In the presence of the covering element, the user cannot see the keypad, for which reason he/she must remove it to be able to freely use the remote control; in this way, the user is certain that the region previously covered by the covering element has not been handled by a previous user.

The known solution described in the quoted Italian patent application does not, however, solve the problem of ensuring hygiene in the use of a remote control adapted for being used by a plurality of users; indeed, such a known solution ensures the hygiene only of the region of the wrapper over the keypad and protected by the covering element, whereas the rest of the wrapper remains subject to the risk of contamination. Indeed, it is possible for a first user to handle the remote control, for example to move it, even without using it to control a device, and thus without having to remove the covering element; in this case, a second user that had to use the remote control, even by removing the covering element would still come into contact with possible germs, bacteria, viruses, or other pathogens deposited on the region of the wrapper not previously covered by the covering element, the removal of which would thus be ineffective in order to protect the second user. The risk of contamination is also accentuated by the fact that the area of the case not covered by the covering element must in any case be handled, for example by a cleaner of the building in which the remote control is used, to insert the remote control in the case itself.

The purpose of the present invention is to avoid the aforementioned drawbacks, and in particular to obtain a hygienical case for remote controls that can guarantee to the user of a remote control a use in hygienic conditions, even in the case in which such a case has already been handled previously.

Another purpose of the present invention is to provide a hygienical case for remote controls that is easy to use.

A further purpose of the present invention is to make a hygienical case for remote controls that can be used in combination with different types of remote control. These and other purposes according to the present invention are accomplished by making a hygienical case for remote controls, comprising a first wrapper adapted to cover totally, or substantially totally, a remote control, and a second wrapper fixed externally to the first wrapper so as to cover totally, or substantially totally, the same, such a second wrapper being permanently removable from the first wrapper. Advantageously, before the removal thereof, the second wrapper covers totally, or substantially totally, the first wrapper, in particular the external surface thereof, preferably like a “shell” or “skin”.

The second wrapper, totally or substantially totally covering the first wrapper, in particular the external surface thereof, prevents the external surface of the first wrapper coming into contact with germs, viruses, bacteria or other pathogens before the removal of such a second wrapper.

The second wrapper, which completely covers the first, and which can be totally removed from the latter, ensures the perfect hygiene of the solution; indeed, irrespective of who might have previously handled the case, it is sufficient for a user to remove the second wrapper to be able to use the remote control, closed inside the first wrapper, in conditions of absolute hygiene. Indeed, the second wrapper, until it is removed, completely protects the first wrapper from germs, viruses, bacteria and other pathogens that the external surface of the second wrapper might have previously come into contact with, thus ensuring the total hygiene of the underlying first wrapper. It should be emphasised that in the present patent application, the expression “substantially totally cover” is meant to indicate cover the totality of an object (for example a remote control, a wrapper), with the possible exclusion (for example due to holes or openings of very small size, in particular less than one millimetre) of regions thereof that are so small as not to constitute a risk for the passage of large amounts of germs, viruses, bacteria or possible pathogens.

Advantageously, before the removal thereof, the second wrapper totally covers the first wrapper like a “shell” or “skin”.

Advantageously, the second wrapper cannot be fixed back onto the first wrapper after the second wrapper has been removed from the first wrapper.

In this way, the risk of the second wrapper being able to be removed and then reapplied to the first wrapper, for example by mistake, after it has been contaminated, is avoided.

Advantageously, the second wrapper can be fixed to the first wrapper by gluing and/or welding.

In a preferred embodiment, the second wrapper is made with a structure and/or from a material such that the removal thereof causes a permanent deformation and/or breaking and/or mechanical and/or aesthetic deterioration thereof, so as to make such a second wrapper unable to be reused.

In a preferred embodiment, the second wrapper can be made from a layer or film (or plurality of layers and/or films) that totally covers the external surface of the first wrapper (like a “shell” or “skin”); preferably, such a layer or film is fixed to the external surface of the first wrapper, for example by a removable adhesive, the removal of which preferably causes a permanent deformation and/or breaking and/or mechanical and/or aesthetic deterioration of the layer or film, so as to prevent it from being subsequently fixed back onto the first wrapper, or more generally reused.

In a further advantageous embodiment, the second wrapper can be fixed to the first wrapper by a connecting element. In this case, preferably, the permanent removal of the second wrapper from the first wrapper takes place by permanent breaking of the connecting element.

Preferably, the possible connecting element has a weakened structure, adapted for facilitating the breaking thereof.

In a preferred embodiment the possible connecting element is made in one piece (i.e. in a single body) with the first wrapper and the second wrapper.

Preferably, the connecting element and the first and the second wrapper are made by co-moulding. Advantageously, the first wrapper is made from plastic material of suitable density so as not to allow germs or bacteria to pass.

Preferably, the first wrapper can be made from polyethylene (PE), or from another plastic material, of the biodegradable, recyclable non-biodegradable or non-recyclable type.

Advantageously, the second wrapper is made from plastic material of suitable density so as not to allow germs or bacteria to pass.

Preferably, the second wrapper can be made from polyethylene (PE), or another plastic material, of the biodegradable, recyclable non-biodegradable or non-recyclable type.

In a preferred embodiment, the second wrapper is impregnated/treated with a disinfecting substance, like for example the substance known by the trade name “AMUCHINA”.

In a preferred embodiment, the case has a envelope-like configuration, more preferably having a rectangular plan, possibly with bevelled or rounded borders.

Advantageously, the case has a flexible, and preferably, but not necessarily, elastically deformable structure, so as to better adapt to different types and sizes of remote control.

In a preferred embodiment, the first wrapper has a envelope-like configuration, more preferably having a rectangular plan, possibly with bevelled or rounded borders.

Preferably, the first wrapper has a flexible, and preferably, but not necessarily, elastically deformable structure, so as to better adapt to different types and sizes of remote control.

In a preferred embodiment, the second wrapper has a envelope-like configuration, more preferably having a rectangular plan, possibly with bevelled or rounded borders, arranged to totally, or substantially totally cover the first, like a “shell” or “skin”. Preferably, the second wrapper has a flexible, and preferably, but not necessarily, elastically deformable structure, so as to better adapt to different types and sizes of remote control.

In a further advantageous embodiment, the second wrapper comprises one or more layers or films fixed removably to the external surface of the first wrapper, completely cover the same like a “shell” or “skin”.

Advantageously, the case comprises an access opening for a remote control. Preferably, the case comprises a closing system adapted for totally, or substantially totally, closing such an access opening. In a preferred embodiment, such a closing system comprises an adhesive and/or a zipper, with or without a cursor. More preferably, such a closing system comprises a layer of adhesive positioned on an inner surface of the first wrapper, near the border of the access opening, adapted for allowing such a border to be fastened in closed position, so as to permanently close the access opening. Preferably, the case comprises a removable protective element of the layer of adhesive, adapted for preventing the closing by gluing of the access opening before the removal of such a removable protective element. Preferably, the removable protective element comprises a strip of plastic material.

In the advantageous case in which the case has a envelope-like structure, the access opening can preferably be formed at a perimeter end of such an envelope.

In a further advantageous embodiment, the second wrapper can be fixed to the first wrapper by means of a removable region of such a second wrapper, fixed to cover the external surface of the first wrapper, for example by means of a removable adhesive, or by welding, preferably at the border of the access opening.

In a preferred embodiment, the case comprises a gripping element, preferably positioned on said second wrapper, to facilitate the removal of the second wrapper from the first wrapper. More preferably, such a gripping element comprises a tongue that projects from the external surface of the second wrapper.

Advantageously, the first wrapper comprises a transparent region adapted to lay over a keypad of a remote control inserted in it.

Preferably, but not necessarily, the first wrapper is totally transparent.

In a preferred embodiment, the second wrapper comprises at least one obstructing region at least partially laying over the transparent region of the first wrapper, so as to at least partially conceal the keypad of a remote control inserted in the case and/or make it not freely usable while the second wrapper is fixed to the first wrapper.

In a further advantageous embodiment, the possible obstructing region of the second wrapper is opaque or partially opaque, so as to at least partially conceal the keypad of a remote control inserted in the case and/or make it not freely usable while the second wrapper is fixed to the first wrapper.

Preferably, the possible obstructing region of the second wrapper comprises one or more inscriptions and/or images positioned on it, so as to at least partially conceal the keypad of a remote control inserted in the case and/or make it not freely usable while the second wrapper is fixed to the first wrapper.

Preferably, the possible one or more inscriptions and/or images contain at least one instruction concerning the removal of the second wrapper.

In a further advantageous embodiment, the possible obstructing region of the second wrapper has an increased rigidity and/or thickness, so as to make the keypad of a remote control inserted in the case not freely usable while the second wrapper is fixed to the first wrapper.

The characteristics and advantages of the hygienical case for remote controls according to the present invention will become clearer from the following description, given as an example and not for limiting purposes, referring to the attached schematic drawings, in which:

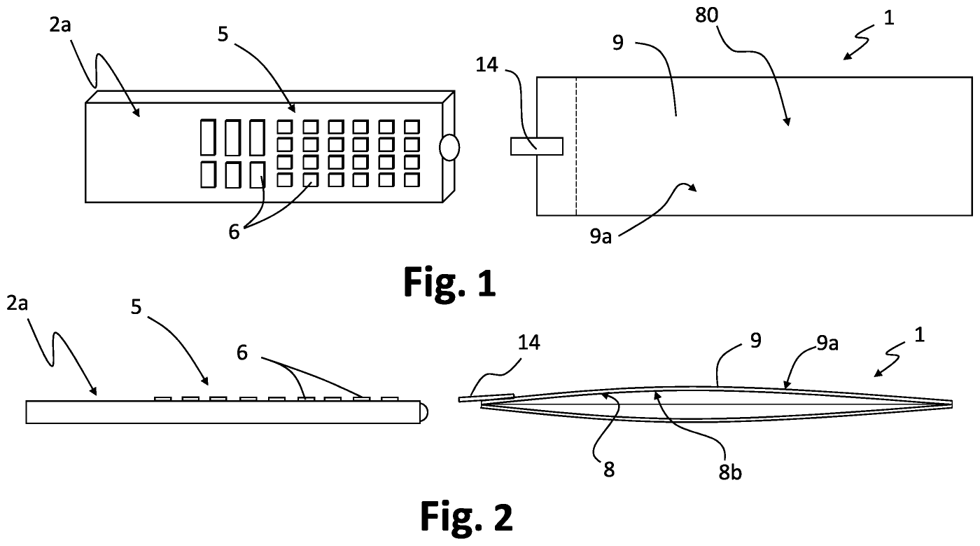

FIG. 1 is a schematic plan view of a remote control5 and of a case according to the invention before the insertion of the remote control in the case;

FIG. 2 is a side view of the remote control and of the case of FIG. 1;

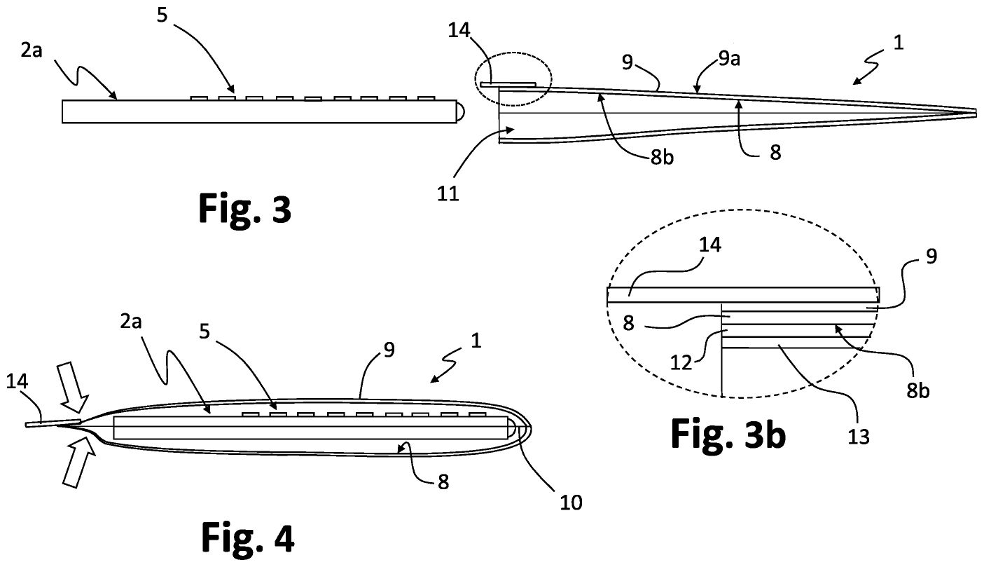

FIG. 3 is a side view of the remote control and of the case of FIG. 1 during the insertion of the remote control in the case;

FIG. 3b is an enlarged detail of FIG. 3;

FIG. 4 is a side view of the remote control and of the case of FIG. 1, with the remote control totally inserted in the case;

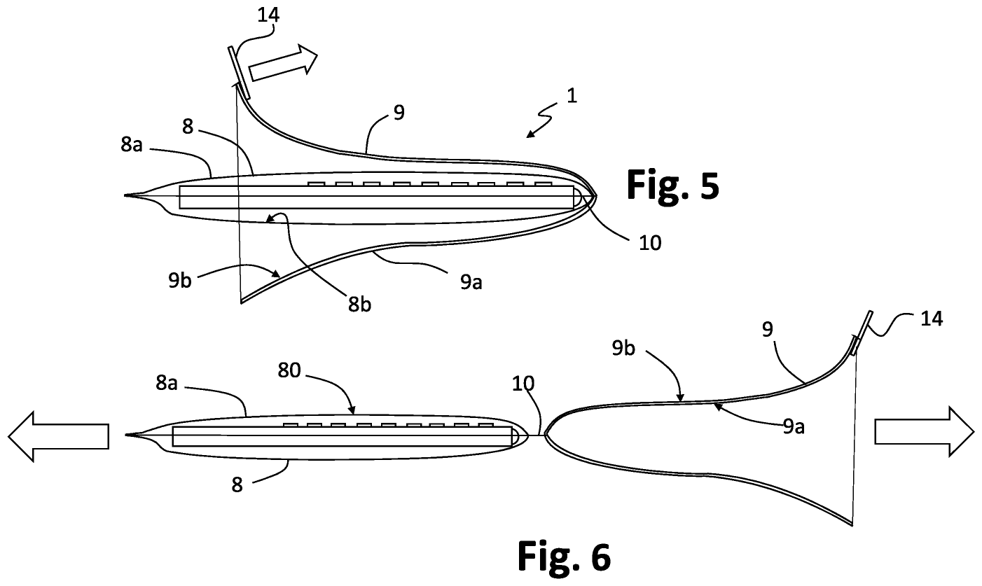

FIG. 5 is a side view of the remote control and of the case of FIG. 1 during the removal of the second wrapper from the first wrapper;

FIG. 6 is a side view of the remote control and of the case of FIG. 1 during a further step of the removal of the second wrapper from the first wrapper;

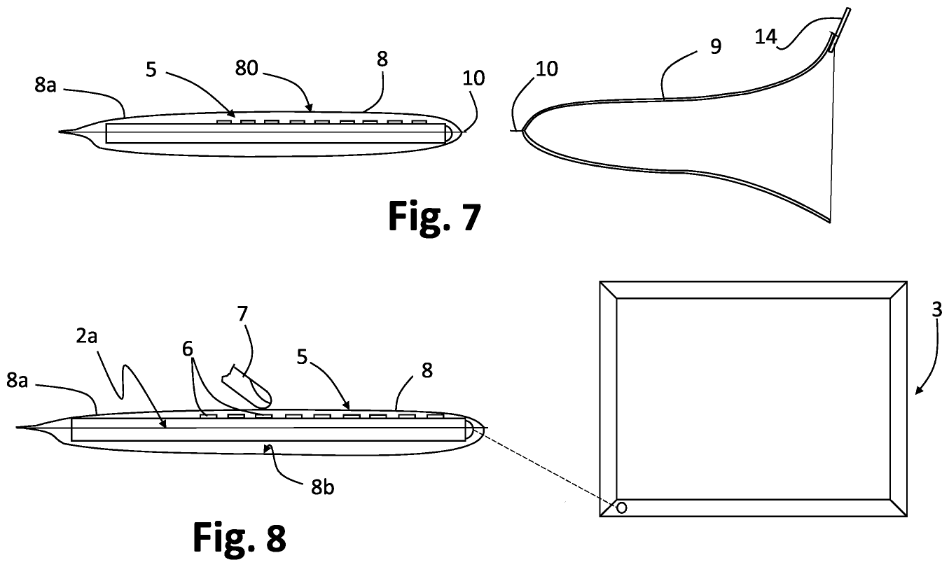

FIG. 7 is a side view of the remote control inserted in the case of FIG. 1 with the second wrapper totally removed;

FIG. 8 is a side view of the remote control and of the case of FIG. 1 during the use of the remote control;

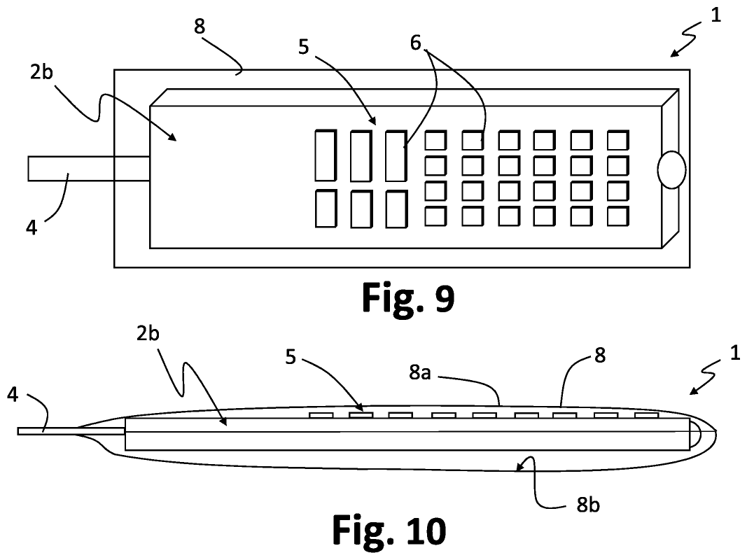

FIG. 9 is a schematic plan view of a wired remote control inserted in a case according to the invention with the second wrapper removed;

FIG. 10 is a side view of the remote control and of the case of FIG. 9;

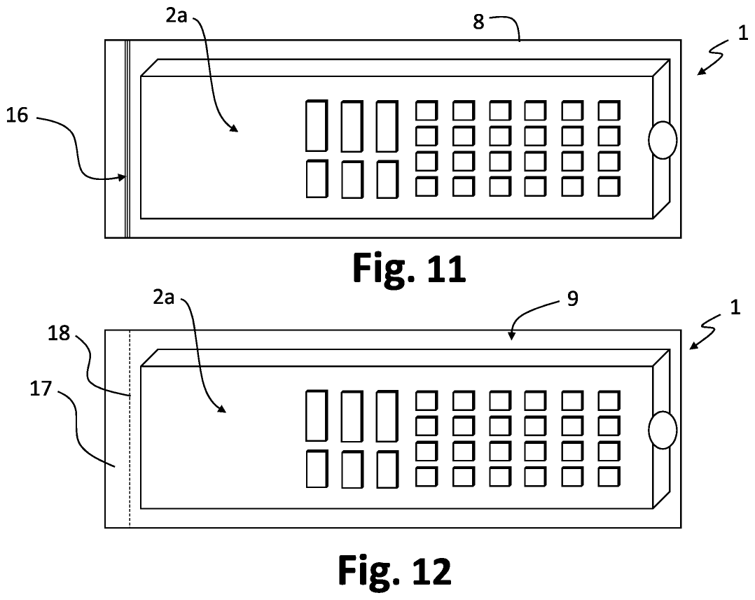

FIG. 11 is a schematic plan view of a remote control inserted in a further embodiment of a case according to the invention;

FIG. 12 is a schematic plan view of a remote control inserted in a further embodiment of a case according to the invention, before the removal of the second wrapper;

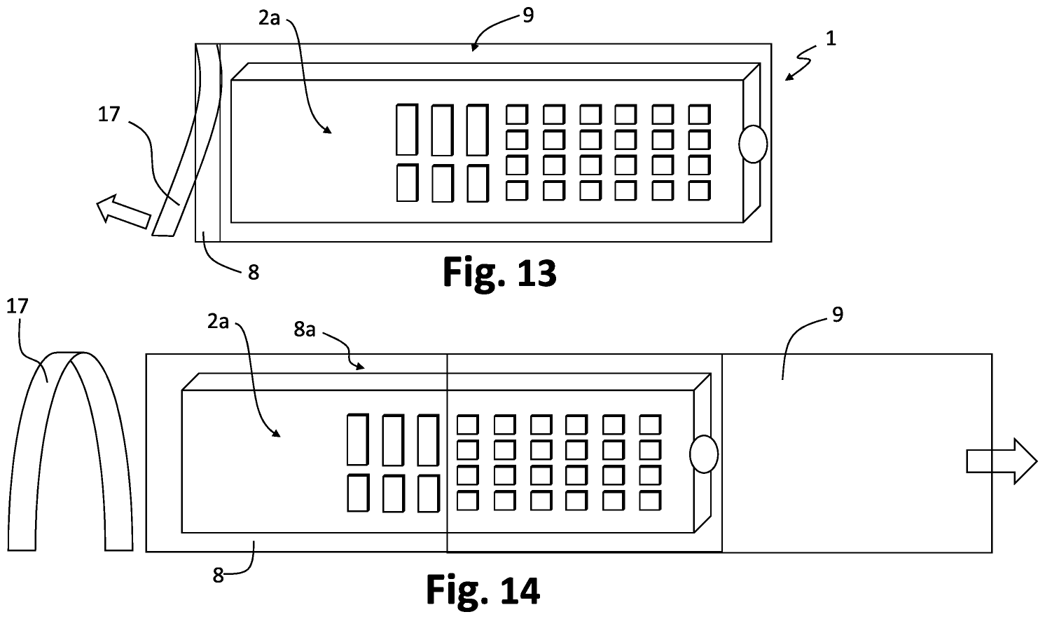

FIGS. 13, 14 and 15 are schematic plan views of the remote control and of the case of FIG. 12, in various steps of the removal of the second wrapper;

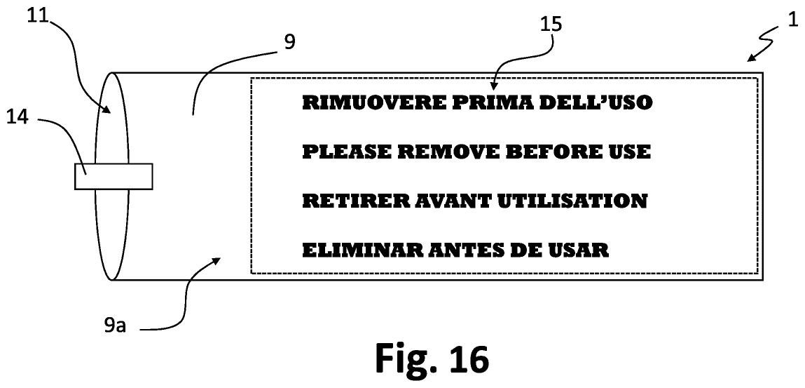

FIG. 16 is a schematic plan view of a further variant embodiment of a case according to the invention.

With reference to the attached figures, a hygienical case for remote controls is wholly indicated with numeral 1; FIGS. 1 to 8, and 11 to 15, represent a first example of remote control, indicated with numeral 2a, of the wireless type, able to be used to remotely control an electric or electronic device. In the non-limiting example of FIG. 8, the electronic device able to be controlled remotely by the remote control 2a is a television set 3, but in a different embodiment, not represented, it could for example be a radio, a computer, a tilting bed, a device for opening/closing curtains, for controlling air conditioners, etc.

In a further embodiment, illustrated for example in FIGS. 9 and 10, the remote control, indicated with numeral 2b, can advantageously be of the wired type, i.e. equipped with a cable 4 that connects it to an electric or electronic device, not represented, to be controlled through the remote control 2b.

In any case, it is emphasised that the invention can be used both with a wireless remote control and with a wired remote control.

The remote control, both in its wireless version 2a, and in its wired version 2b, preferably comprises a keypad 5, comprising one or more keys 6, preferably of the electromechanical and/or “touch” type, by acting on which, for example pressing them, a user, whose finger is represented schematically in FIG. 8, can activate one or more functions of the device controlled remotely by the remote control; for example, in the case in which such a device is a television set 3, the keypad 5 can allow the channel to be changed, the volume to be increased, etc.

In the case in which, for example, the remote control is intended for controlling the tilt of a bed, the keypad 5 can comprise keys for controlling such tilt. The case 1 comprises a first wrapper 8 adapted for totally, or substantially totally, housing the remote control 2a, or 2b.

Advantageously, the first wrapper 8 comprises at least one transparent region 80 adapted for laying over the keypad 5 of a remote control 2a, 2b inserted in it, so as to allow it to be seen by a user.

Advantageously, but not necessarily, the first wrapper is completely transparent.

The case 1 comprises a second wrapper 9 fixed externally to the first wrapper 8 so as to totally, or substantially totally, cover the same, and permanently removable from such a first wrapper 8.

Advantageously, before the removal thereof, the second wrapper 9 totally covers the first wrapper 8, in particular the external surface 8a thereof, like a “shell” or “skin”.

Advantageously, as will be explained better hereinafter, the second wrapper 9 cannot be fixed back to the first wrapper 8 once the second wrapper 9 has been removed from the first wrapper 8.

Advantageously, the first wrapper 8 is made from plastic material of suitable density so as not to allow germs or bacteria to pass (for example it can have a thickness comprised between 0.005 mm and 1 mm); preferably the first wrapper 8 is made from polyethylene (PE), or another plastic material, of the biodegradable, recyclable non-biodegradable or non-recyclable type.

Advantageously, the second wrapper 9 is also made from plastic material of suitable density so as not to allow germs or bacteria to pass (for example it can have a thickness comprised between 0.005 mm and 1 mm), preferably polyethylene (PE), or another plastic material, of the biodegradable, recyclable non-biodegradable or non-recyclable type. Advantageously, but not necessarily, the second wrapper 9 is impregnated or treated with a disinfecting substance, like for example the substance known by the trade name “AMUCHINA”.

In a preferred embodiment, the second wrapper 9 is fixed to the first wrapper 8 by gluing and/or welding. For example, the second wrapper 9 can be made from a layer or film that totally covers the external surface 8a of the first wrapper 8 (like a “shell” or “skin”); such a layer or film is fixed to such an external surface 8a, for example, through a removable adhesive, the removal of which causes a permanent deformation and/or breaking and/or mechanical and/or aesthetic deterioration of the layer or film itself, so as to prevent it from subsequently being fixed back to the first wrapper 8, or, more generally, reused.

In a further advantageous embodiment, represented for example in FIGS. 1 to 8, the second wrapper 9 can be fixed to the first wrapper 8 by a connecting element 10. Advantageously, the connecting element 10 can be provided, alternatively or in combination, with a further fixing means, like for example gluing. For example, the second wrapper 9 can be made from a layer or film that totally covers the external surface 8a of the first wrapper 8, such a layer or film being fixed to such an external surface 8a through a removable adhesive, and being further fixed to the first wrapper 8 by a connecting element 10, for example a flap of plastic material that joins the first wrapper 8 to the second wrapper 9.

Preferably, the connecting element 10 joins the external surface 8a of the first wrapper 8 to the inner surface 9b of the second wrapper 9, and is positioned so as to be totally covered by the second wrapper 9 when it totally covers the first wrapper 8, so that the second wrapper 9 also protects the connecting element 10 from contact with germs, viruses, bacteria, or other pathogens that might come into contact with the external surface 9a of the second wrapper 9 before it has been removed.

Advantageously, the permanent removal of the second wrapper 9 from the first wrapper 8 takes place through permanent breaking of the connecting element 10. For example, with reference to FIGS. 6 and 7, by exerting a traction of the second wrapper 9 in the direction away from the first wrapper 8, in order to permanently remove such a second wrapper 9 from the first wrapper 8, once the ultimate tensile strength of the connecting element 10 has been exceeded, the latter breaks, with consequent permanent removal of the second wrapper 9 from the first wrapper 8.

Preferably, but not necessarily, the connecting element 10 has a weakened structure, adapted to facilitate the breaking thereof; for example, the connecting element 10 can have tear perforations obtained with a series of aligned holes that pass through it, reducing the resistance thereof to tearing.

Preferably, but not necessarily, the connecting element 10 is made in one piece with the first wrapper 8 and with the second wrapper 9, for example through co-moulding of such three elements, or through over-moulding of the connecting element 10 on the first and second wrapper, to connect them together.

Advantageously, the case 1 has a envelope-like configuration, preferably with a rectangular plan, possibly with bevelled or rounded borders.

Preferably, the case 1 has a flexible, and advantageously, but not necessarily, elastically deformable structure, so as to better adapt to different types and sizes of remote control. In a preferred embodiment, the first wrapper 8 has a envelope-like configuration, more preferably with a rectangular plan, possibly with bevelled or rounded borders.

Preferably the first wrapper 8 has a flexible, and preferably, but not necessarily, elastically deformable structure, so as to better adapt to different types and sizes of remote control.

In a preferred embodiment, the second wrapper 9 has a envelope-like configuration, more preferably with a rectangular plan, possibly with bevelled or rounded borders, arranged to totally, or substantially totally cover the first wrapper, like a “shell” or “skin”.

Preferably, the second wrapper 9 has a flexible, and preferably, but not necessarily, elastically deformable structure, so as to better adapt to different types and sizes of remote control.

In an advantageous variant embodiment, not represented in the attached figures, the second wrapper 9 can comprise one or more layers or films removably fixed to the external surface 8a of the first wrapper 8, to totally, or substantially totally, cover the same.

Advantageously, the case 1 comprises an access opening 11 for the insertion of the remote control 2a, 2b.

In the case in which the case 1 has a envelope-like structure, the access opening 11 can be advantageously formed at a perimeter end of such an envelope.

In a further advantageous embodiment, not represented, the access opening 11 can be formed in a different position of the case 1.

Preferably, the case 1 comprises a closing system adapted for totally, or substantially totally, closing the access opening 11.

It is emphasised that in the present patent application, the expression “substantially totally close the access opening” means completely closing the opening, with the possible exclusion of regions thereof that are so small (preferably of maximum dimensions of less than one millimetre) as not to constitute a risk for the passage of large amounts of germs, viruses, bacteria or possible pathogens.

Advantageously, the closing system of the access opening 11 can comprise an adhesive and/or a zipper, of the type with or without a cursor.

For example, in the example embodiment illustrated schematically in FIG. 3b, such a closing system can advantageously comprise a layer of adhesive 12 positioned on an inner surface 8b of the first wrapper 8, near to the border of the access opening 11; the layer of adhesive 12 allows such a border to be fastened in closed position (as shown for example in FIG. 4), so as to permanently close the access opening 11.

Advantageously, the case 1 can comprise a removable protective element 13 of the layer of adhesive 12, adapted to prevent the closing by gluing of the access opening 11 before the removal of such a removable protective element 13.

Advantageously, the removable protective element 13 can comprise a strip of plastic material.

In the example embodiment illustrated in FIGS. 9 and 10, the border of the access opening 11, thanks to the flexibility of the case 1, can be closed on itself to wrap around the cable 4, allowing it to come out from the case 1 and at the same time ensuring that the remote control 2b is totally, or substantially totally, closed in the case 1.

In a further embodiment, represented schematically in FIG. 11, the closing system of the access opening 11 can comprise a zipper 16 arranged at such an access opening 11 on the first wrapper 8, or any known fixing means. In the example embodiment of FIG. 11, the zipper 16 is not provided with a cursor, but in a further advantageous embodiment, not represented, the zipper 16 can be provided with a cursor, adapted to facilitate the closing thereof.

In a further advantageous embodiment, represented schematically in FIGS. 12 to 15, the second wrapper 9 can be fixed to the first wrapper 8 through a removable region 17 of such a second wrapper, fixed to cover the external surface 8a of the first wrapper 8, for example through a removable adhesive, advantageously at the border of the access opening 11.

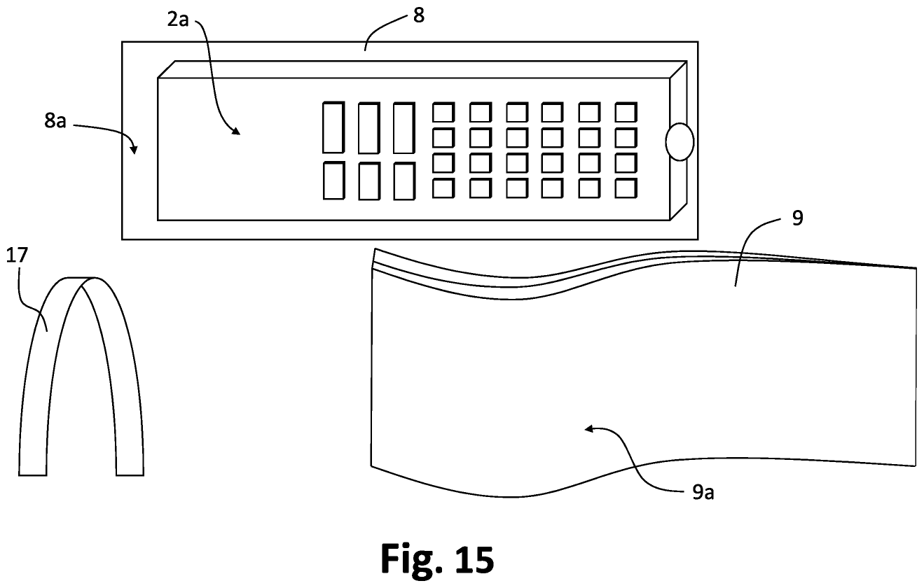

As illustrated schematically in FIGS. 12 to 15, in such an advantageous embodiment, once a remote control, for example of the wireless type 2a (but the same is also valid in the case of a wired remote control 2b), has been inserted in the case 1, and the access opening 11 thereof has been closed through the closing system provided (for example an adhesive and/or a zipper, with or without a cursor) the removable region 17 of the second wrapper 9 can be removed from the rest of the second wrapper 9 itself, for example tearing it at a weakened region 18, and separating it from the external surface 8a of the first wrapper 8, for example by pulling. At this point, as illustrated for example in FIGS. 14 and 15, the second wrapper 9 can be removed, leaving the remote control 2a protected by the first wrapper 8.

Advantageously, the case 1 can comprise a gripping element adapted to facilitate the removal of the second wrapper 9 from the first wrapper 8.

Preferably, the gripping element comprises a tongue 14 that projects from the external surface 9a of the second wrapper 9.

In an advantageous variant embodiment, represented for example in FIG. 16, the second wrapper 9 comprises at least one obstructing region 15 at least partially laying over the transparent region 80 of the first wrapper 8, so as to at least partially conceal the keypad 5 of a remote control 2a, 2b inserted in the case 1 and/or make it not freely usable while such a second wrapper 9 is fixed to the first wrapper 8.

In an advantageous embodiment, the obstructing region of the second wrapper 9 is opaque or partially opaque, so as to at least partially conceal the keypad 5 of a remote control 2a, 2b inserted in the case 1 and/or make it not freely usable while such a second wrapper 9 is fixed to the first wrapper 8.

Preferably, but not necessarily, the obstructing region 15 comprises one or more inscriptions and/or images positioned on the same, so as to at least partially conceal the keypad 5 of a remote control 2a, 2b inserted in the case 1 and/or make it not freely usable while such a second wrapper 9 is fixed to the first wrapper 8.

Preferably, such one or more inscriptions and/or images contain at least one instruction concerning the removal of the second wrapper 9; such instructions can comprise, for example, the indication that the second wrapper 9 must be removed before the remote control is used.

In a further advantageous embodiment, the obstructing region 15 has an increased rigidity and/or thickness, so as not to make the keypad 5 of a remote control 2a, 2b inserted in the case 1 freely usable (for example because the increased thickness of the obstructing region 15 makes the keypad unable to be freely handled) while the second wrapper 9 is fixed to the first wrapper 8; such increased rigidity and/or thickness can be obtained, for example, through the positioning of an insert made of rigid plastic material inside or above the second wrapper 9.

In a further advantageous variant embodiment, not represented, irrespective of whether there is an obstructing region 15, the first wrapper 8 and/or the second wrapper 9 can carry one or more inscriptions and/or drawings, for example showing a trade name or logo and/or operating instructions for using the case 1, like for example the instructions of removing the second wrapper 9 before use, or the indication to request a new case 1 in the case in which the second layer is not present (for example because it has already been removed by a previous user).

The operation of the case 1 according to the invention is as follows.

Whenever the structure at which the remote control is installed is cleaned and prepared to receive a new user, the person tasked with cleaning and/or preparation removes a possible case already present on a remote control 2a, 2b, and inserted the latter in a case 1 according to the invention, through the access opening 11, making sure that the remote control 2a, 2b is totally, or substantially totally, closed in the case 1.

The access opening 9 is then closed through the closing system present in the case 1, for example, with reference to FIGS. 1-10, removing the protective element 13 and then closing the border of the access opening 11 on the layer of adhesive 12, so as to seal the border of the access opening 11 in closed condition. In the further embodiment illustrated in FIG. 11 the access opening can be closed by acting on the zipper 16.

In such a condition, the remote control 2a, 2b is totally closed by the case 1; in the case in which the remote control 2b is of the wired type, only the cable 4 comes out from the case 1.

When a new user is about to use the remote control 2a, 2b closed in the case 1, before using it removes, for example by acting on the gripping element 14, the second wrapper 9, which previously totally closed the first wrapper 8 and the remote control 2a, 2b.

Once removed, the second wrapper 9 cannot be fixed back to the first wrapper 8, for example because it has deteriorated during its removal and/or because a possible connecting element 10 that joined it to the first wrapper 8 has been broken during the removal of the second wrapper 9.

Before the removal of the second wrapper 9, the external surface 8a of the underlying first wrapper 8 was protected by the presence of the second wrapper 9, so that possible handling of the case prior to the removal of the second wrapper 9 does not entail the risk of the external surface 8a of the first wrapper 8 coming into contact with possible germs, bacteria, viruses, or other pathogens.

In the advantageous, but not necessary, case in which the second wrapper 9 has an obstructing region 15, the user is forced to remove the second layer 9 to be able to freely use the remote control; in this way, the risk of the user forgetting to remove the second layer 9 before use is reduced.

From the description that has been made the characteristics of the device object of the present invention are clear, just as the relative advantages are also clear.

Finally, it is clear that the device thus conceived can undergo numerous modifications and variants, all encompassed by the invention; moreover, all of the details can be replaced by technically equivalent elements. In practice, the materials used, as well as the sizes, can be whatever according to the technical requirements.

1. A hygienical case for remote controls, comprising:
a first wrapper adapted to cover totally, or substantially totally, a remote control, and
a second wrapper fixed externally to the first wrapper so as to cover totally, or substantially totally, the first wrapper,
wherein the second wrapper is permanently removable from s-aid the first wrapper.

2. The hygienical case of claim 1, wherein the second wrapper is fixed to the first wrapper by gluing and/or welding.

3. The hygienical case of claim 1, wherein the second wrapper is fixed to the first wrapper by a connecting element.

4. The hygienical case of claim 3, wherein the permanent removal of the second wrapper from the first wrapper takes place by permanent breaking of the connecting element.

5. The hygienical case of claim 1, wherein the second wrapper is impregnated and/or treated with a disinfecting substance.

6. The hygienical case of claim 1, wherein the second wrapper comprises one or more layers or films fixed removably to an external surface of the first wrapper, to completely cover the first wrapper.

7. The hygienical case of claim 1, further comprising an access opening for a remote control.

8. The hygienical case of claim 7, further comprising a closing system adapted to totally, or substantially totally, close the access opening.

9. The hygienical case of claim 8, wherein the closing system comprises a layer of adhesive positioned on an inner surface of the first wrapper, near a border of the access opening, the layer of adhesive adapted to allow the border to be fastened in a closed position, so as to permanently close the access opening, or
wherein the closing system comprises a zipper with or without a cursor.

10. The hygienical case of claim 1, further comprising a gripping element to facilitate the removal of the second wrapper from said the first wrapper.
